(19) United States
(12) Patent Application Publication
(10) Pub. No.: US 2025/0331833 A1
(43) Pub. Date: Oct. 30, 2025

(54) HEMOSTASIS DEVICES AND METHODS OF USE

(71) Applicant: Merit Medical Systems, Inc., South Jordan, UT (US)

(72) Inventors: Richard Stephenson, Salt Lake City, UT (US); Aaron Hopkinson, Herriman, UT (US)

(21) Appl. No.: 19/188,717

(22) Filed: Apr. 24, 2025

Related U.S. Application Data
(63) Continuation of application No. 17/806,653, filed on Jun. 13, 2022.
(60) Provisional application No. 63/212,227, filed on Jun. 18, 2021.

(51) Int. Cl. A61B 17/00; A61B 17/12; A61B 17/135

(52) U.S. Cl. CPC A61B 17/0057; A61B 17/135; A61B 2017/00659; A61B 2017/12004

(57) ABSTRACT

A hemostasis device configured to apply a compressive force to a puncture site of a patient's vessel, such as an artery of the foot, is disclosed. The hemostasis device may include a compression member having a plurality of tabs and an inflatable bladder. A plurality of bands may be used to position and maintain the compression member over the puncture site until hemostasis is achieved are releasably coupled to the plurality of tabs. A first band can wrap around the ankle, a second band can wrap around the foot, and a third band can extend between two toes or around a toe. The hemostasis device may also comprise an inflation port in fluid communication with the inflatable bladder.

### RELATED APPLICATIONS

This is a continuation of U.S. patent application Ser. No. 17/806,653, filed on Jun. 13, 2022 and titled, “Hemostasis Devices and Methods of Use,” which claims priority to U.S. Provisional Application No. 63/212,227, filed on Jun. 18, 2021 and titled, “Hemostasis Devices and Methods of Use,” both of which are hereby incorporated by reference in their entireties.

### TECHNICAL FIELD

The present disclosure relates generally to the field of medical devices used to provide compression or hemostasis at a vascular access puncture site. More particularly, some embodiments of the present disclosure relate to a hemostasis device used to provide hemostasis of the arteries of the lower leg and foot, including the anterior tibial artery, dorsalis pedis artery, posterior tibial artery, and peroneal artery, following vascular access.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a top view of an embodiment of a hemostasis device.

FIG. 2A is a top, exploded view of the hemostasis device of FIG. 1.

FIG. 2B is a cross-sectional view of the hemostasis device of FIG. 1 taken through section 2B-2B, with an inflatable bladder inflated.

FIG. 3A is perspective view of the hemostasis device of FIG. 1 in a use configuration.

FIG. 3B is a perspective view of the hemostasis device of FIG. 1 in an alternative use configuration.

FIG. 4 is a top view of another embodiment of a hemostasis device.

FIG. 5 is a top, exploded view of the hemostasis device of FIG. 4.

FIG. 6 is a perspective view of the hemostasis device of FIG. 4 in a use configuration.

FIG. 7A is a top perspective view of the hemostasis device of FIG. 4 in an alternative use configuration.

FIG. 7B is a bottom perspective view of the hemostasis device of FIG. 4 in the alternative use configuration.

### DETAILED DESCRIPTION

Interventional vascular procedures may be performed for both diagnostic and therapeutic reasons. Some vascular interventional procedures include percutaneous access of a vessel, such as an artery, with a needle followed by insertion of guidewires, dilators, sheaths, catheters, stents, balloons, etc. In some procedures, the vessel to be accessed may be a peripheral artery of the leg or foot. For example, the peripheral artery may be any one of the anterior tibial artery, the dorsalis pedis artery, the posterior tibial artery, and the peroneal artery. The vessel access creates a puncture site or hole in the vessel wall through which medical devices may be passed during a procedure. For example, the vessel access may facilitate procedures such as retrograde interventions for advanced peripheral arterial disease and critical limb ischemia of the lower leg. Additionally, the vessel access can allow genicular artery embolization for relief of long-term pain of the knee. At the conclusion of the procedure, the procedural devices may be removed and pressure or compression applied to the puncture site until hemostasis is achieved. The compression may be applied by a healthcare worker or by a hemostasis device, such as the hemostasis devices described herein. Such hemostasis devices may comprise bands for securement of the device to a foot of the patient and an inflatable compression member to apply a pressure or compressive force to the puncture site until hemostasis is achieved.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

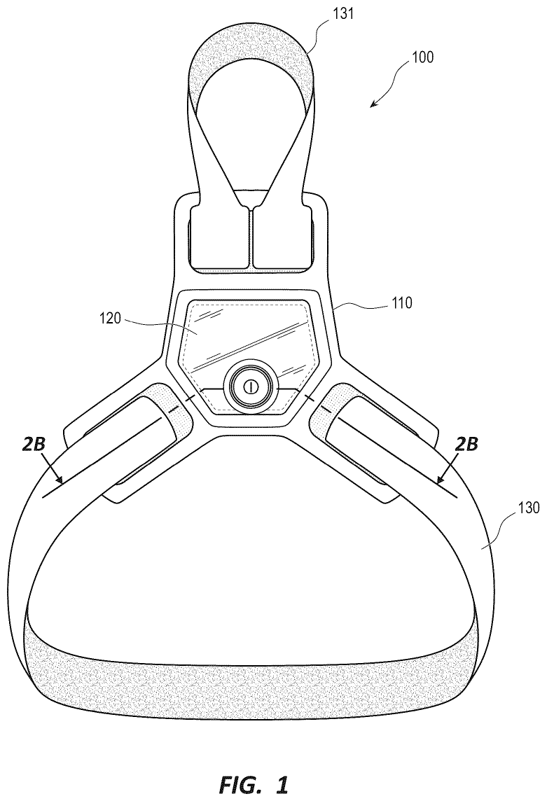

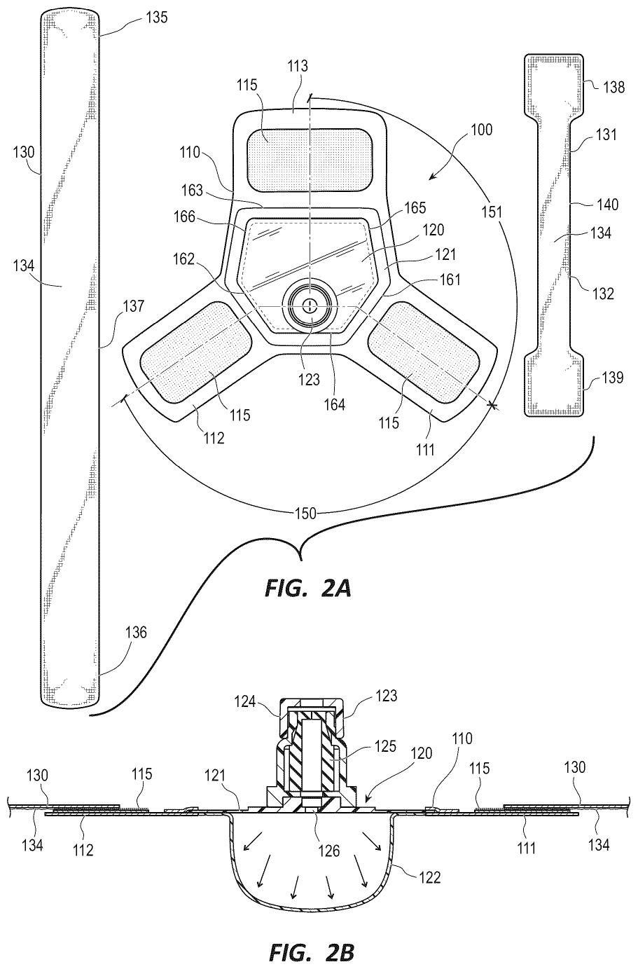

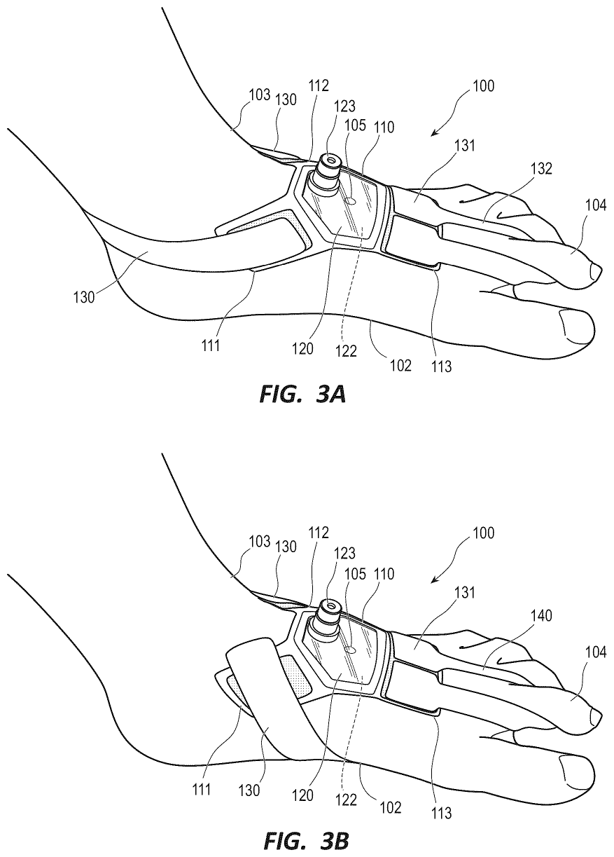

FIG. 1 provides a top view of a vascular access hemostasis device, system, or assembly 100. FIG. 2A provides a top, exploded view of the hemostasis device 100. FIG. 2B provides a cross-sectional view of the hemostasis device 100 with an inflatable bladder inflated. FIG. 3A provides a perspective view of the hemostasis device 100 in a use configuration. FIG. 3B provides a perspective view of the hemostasis device 100 in an alternative use configuration.

As illustrated in the embodiment of FIG. 1, the hemostasis device 100 includes a body 110, a compression member 120, an ankle band or strap 130, and a toe band or strap 131. The hemostasis device 100 may be configured to fit around a lower limb, such as a foot and a toe, to provide compression to a vascular access puncture site in the vasculature, including at any one of the anterior tibial artery, the dorsalis pedis artery, the posterior tibial artery, and the peroneal artery. Other vascular access sites are contemplated. The hemostasis device 100 may be configured to be a left lower limb device or a right lower limb device such that the hemostasis device 100 may provide a hemostasis puncture site in the left foot or right foot.

With reference to FIGS. 2A and 2B, the body 110 is shown to include a first coupling tab 111, a second coupling tab 112, and a third coupling tab 113. The body 110 may be formed from a flexible material that is conformable to a contour of an anatomical member, such as a foot. For example, the body, may be comprised of polyethylene, polypropylene, polyvinyl chloride, etc. Each of the coupling tabs 111, 112, 113 extend radially outward from a central portion of the body 110. Each of the coupling tabs 111, 112, 113 may include a releasable coupling member 115, such as a “hook” portion of a “hook-and-loop” material disposed on an upper surface. Other types of releasable securement members, such as pressure sensitive adhesives, buckles, magnets, snaps, clasps, etc. are contemplated to be within the scope of this disclosure.

The coupling tabs 111, 112, 113 are circumferentially disposed around a perimeter of the body 110. In the illustrated embodiment, the body 110 is in a substantially “Y” shape. Other shapes and configurations are contemplated within the scope of this disclosure. An angle 150 between the first coupling tab 111 and the second coupling tab 112 can range from about 60 degrees to about 120 degrees. An angle 151 between the first coupling tab 111 and the third coupling tab 113 can range from about 90 degrees to about 150 degrees.

The ankle band 130 and the toe band 131 may be formed from a flexible material, such as a plastic film, cloth, etc. The bands 130, 131 may comprise a releasable coupling member 134, such as a “loop” portion of a “hook-and-loop” material configured to releasably couple with the releasable coupling member 115. In some embodiments, the “loop” material may be integral to the material of the bands 130, 131. In other embodiments, the “loop” material may be coupled to a surface of the bands 130, 131. The “loop” material may cover a bottom surface of the bands 130, 131 over a full length of the bands 130, 131. In other embodiments, the “loop” material may cover a portion of the bottom surface of the bands 130, 131. Other types of releasable securement mechanisms, such as pressure sensitive adhesives, buckles, magnets, snaps, clasps, etc. are contemplated to be within the scope of this disclosure.

In the illustrated embodiment, the ankle band 130 comprises a first end portion 135, a second end portion 136, and an intermediate portion 137. In the depicted embodiment, the first end portion 135 is releasably couplable to the first coupling tab 111, the second end portion 136 is releasably couplable to the second coupling tab 112, and the intermediate portion 137 is configured to wrap around the patient's ankle such that a length of the ankle band 130 between the first and second coupling tabs 111, 112 is adjustable. Alternatively, the ankle band 130 may be wrapped around the patient's foot dependent upon the location of the puncture site. This configuration facilitates adjustment of the length of the ankle band 130 to fit a range of ankle circumferences. In the illustrated embodiment the ankle band 130 is fully detachable from the body 110. In other embodiments, the ankle band 130 may be fixedly attached to the body 110 at the first end portion 135 and releasably coupled to the body 110 at the second end portion 136 or vice versa. Additionally, embodiments wherein the ankle band 130 is fixedly attached to the body 110 at both ends, including embodiments wherein the ankle band 130 comprises an elastic material, are likewise within the scope of this disclosure.

In the illustrated embodiment, the toe band 131 comprises a first end portion 138, a second end portion 139, and an intermediate portion 140. In the depicted embodiment, the first end portion 138 is releasably couplable to the third coupling tab 113, the second end portion 139 is releasably couplable to the third coupling tab 113, and the intermediate portion 140 is configured to wrap around the patient's toe such that a length of the toe band 131 between the third coupling tab 113 is adjustable. This configuration facilitates adjustment of the length of the toe band 131 to fit a range of distances of the puncture site from the patient's toe. In another configuration, the toe band 131 may extend between adjacent toes of the patient with the first end portion 138 releasably coupled to the third coupling tab 113 and the second end portion 139 releasably coupled to the ankle band 130 at a bottom surface of the patient's foot. In the illustrated embodiment the toe band 131 is fully detachable from the body 110. In another embodiment, the toe band 131 may be fixedly attached to the body 110 at the first end portion 138 and releasably coupled to the body 110 at the second end portion 139 or vice versa. Additionally, embodiments wherein the toe band 131 is fixedly attached to the body 110 at both ends, including embodiments wherein the toe band 131 comprises an elastic material, are likewise within the scope of this disclosure. A width of the intermediate portion 140 may be less than a width of the first and second end portions 138, 139. This configuration may facilitate greater patient comfort as the toe band 131 wraps around or extends between the patient's toes. The toe band 131 may be configured to wrap around or extend between any of the patient's toes.

Additionally, in embodiments wherein one or more ends of the ankle band 130 and/or toe band 131 are releasably coupleable to the coupling tabs 111, 112, 113 (such as by a hook and loop fastener) the angle at which the ankle band 130 and/or toe band 131 couples to the respective coupling tab 111, 112, 113 may be adjustable over a range. During a procedure, a practitioner can couple one or more ends of the ankle band 130 and/or toe band 131 to a coupling tab 111, 112, 113 at an angle positions the body 110 at a desired position as well as the bands at desired positions on the patient's anatomy. Use of releasably coupleable bands 130, 131 may thus facilitate adjustability and conformability of the device to a patient's anatomy and the position of an access site.

With continued reference to FIG. 2A, the compression member 120 may be configured to be positioned over the vascular access puncture site on the foot of a patient. The compression member 120 may be configured to be inflatable as illustrated in the embodiment of FIG. 2B. In some embodiments, the compression member 120 may be configured to provide compression using a mechanical system including rigid compression pads, resilient or elastomeric compression pads, and/or mechanisms configured to displace the pads such as a threaded post. In other embodiments, the compression member 120 may comprise at least one gauze pad. In still other embodiments, the compression member 120 may be configured to compress the vascular access puncture site using any suitable mechanism.

As illustrated in FIGS. 2A and 2B, the compression member 120 comprises a top plate 121, an inflatable bladder 122, and an inflatable port 123. The top plate 121 may be formed of clear or translucent material to allow observation of the puncture site. For example, the top plate 121 may comprise polycarbonate, polystyrene, styrene copolymers, polyethylene teraphthalate, acrylic, polyethylene, polypropylene, etc. The top plate 121 may be sealingly coupled to the body 110. As depicted, the top plate includes a first side 161, a second side 162, a third side 163, a fourth side 164, a fifth side 165, and a sixth side 166. The first side 161 is adjacent the first coupling tab 111, the second side 162 is adjacent the second coupling tab 112, and the third side 163 is adjacent the third coupling tab 113. Other shapes of the top plate 121 are contemplated. The top plate 121 may be formed in a concave shape relative to a top surface such that the top plate 121 conforms to an anatomical shape of the foot.

The inflatable bladder 122 comprises a flexible wall configured to extend downward from the top plate 121, as depicted in FIG. 2B, when the bladder 122 is filled with air or fluid such that a compressive force may be applied to the vascular access puncture site. The bladder 122 may be sealed at its edges to the bottom surface of the top plate 121. The bladder 122 may be transparent or translucent such that the puncture site can be seen through the flexible wall. The bladder 122 may be formed from any suitable, flexible, transparent or translucent material, such as polyethylene, polypropylene, etc. The fluid may comprise sterile water, normal saline solution, or any other suitable medical fluid. In some embodiments, a target may be printed on an exterior or interior service of the wall of the bladder 122. The target may facilitate alignment of the compression member 120 with the vascular access puncture site.

In the illustrated embodiment, the inflation port 123 is coupled to the top surface of the top plate 121 such that the inflation port 123 is in fluid communication with the inflatable bladder 245 through an orifice 126 through the top plate 121. The inflation port 123 is positioned on the top plate 121 toward an outer perimeter of the top plate 121 adjacent the fourth side 164 and between the first side 161 and second side 162 such that the inflation port 123 does not obstruct visualization of the vascular access puncture site.

The inflation port 123 may comprise a valve member 125 and a housing 124 configured to retain the valve member 125. The housing 124 can include a female Luer fitting to facilitate releasably coupling of a medical device, such as a syringe, to the inflation port 123. When coupled to the syringe, the valve member 125 may be axially displaced such that the inflation port 123 is open and air or liquid may be injected into the inflatable bladder 122 to inflate the compression member 120 to provide a compressive force at the vascular access puncture site. Additionally, air or liquid may be removed from the inflatable bladder 122 to deflate the compression member 120 and reduce the compressive force on the vascular access puncture site. In certain embodiments the valve member 125 may comprise a pre-slit septum such that a non-Luer access device coupled to the syringe can penetrate the septum to access the inflation port 123. Additionally, the inflation port 123 may be configured to mate only with a specified inflation device, including embodiments wherein the inflation device is not configured to mate with a standard luer connector, in order to reduce the chance of accidental coupling of the inflation device to other medical devices accessing the patient's body.

In use, in some embodiments, a retrograde procedure may be performed by a healthcare worker on the patient where access to a peripheral artery of the leg, such as the anterior tibial artery, the dorsalis pedis artery, the posterior tibial artery, and the peroneal artery, is necessary due to blockage of arteries of the upper leg. The retrograde procedure may include interventions for advanced peripheral arterial disease and critical limb ischemia of the lower leg. Additionally, the vessel access can allow genicular artery embolization for relief of long-term pain of the knee.

At the conclusion of the retrograde procedure, compressive pressure or force may be applied to the vascular access puncture site of the distal radial artery to facilitate hemostasis of the vascular access puncture site as quickly as possible and without loss of a significant blood volume and with reduced time required from the healthcare worker. Referring to FIGS. 3A and 3B, the hemostasis device 100 may be utilized to apply the compressive pressure to a vascular access puncture site 105. The compression member 120 may be positioned over the vascular access puncture site 105 at a dorsal or medial aspect of a patient's foot 102. As shown in FIG. 3A, the ankle band 130 may be extended around the patient's ankle 103 and be releasably coupled to the first and second coupling tabs 111, 112 to restrict distal movement of the hemostasis device relative to the patient's foot 102. Alternatively, as shown in FIG. 3B, the ankle band 130 may extend around the patient's foot 102 to provide a downward force to the hemostasis device. The toe band 131 may extend toward the patient's toes 104 such that the intermediate portion 140 is wrapped around any one of the patient's toes 104 and may be releasably coupled to the third coupling tab 113 to restrict proximal movement of the hemostasis device 100 relative to the patient's foot 102.

Following placement of the hemostasis device 100, the syringe (not shown) may be coupled to the inflation port 123 and air or liquid injected through inflation port 123 into the inflatable bladder 122 such that the inflatable bladder 122 is inflated and applies a compressive pressure or force to the vascular access puncture site 105. In some embodiments, the syringe may be coupled to the inflation port 123 to remove a portion of the air or liquid within the inflatable bladder 122 such that the inflatable bladder 122 is partially deflated. In other embodiments, the syringe may be coupled to the inflation port 123 to remove all of the air or liquid within the inflation bladder 122 such that the inflatable bladder 122 is fully deflated and the compressive force is relieved from the vascular access puncture site 105.

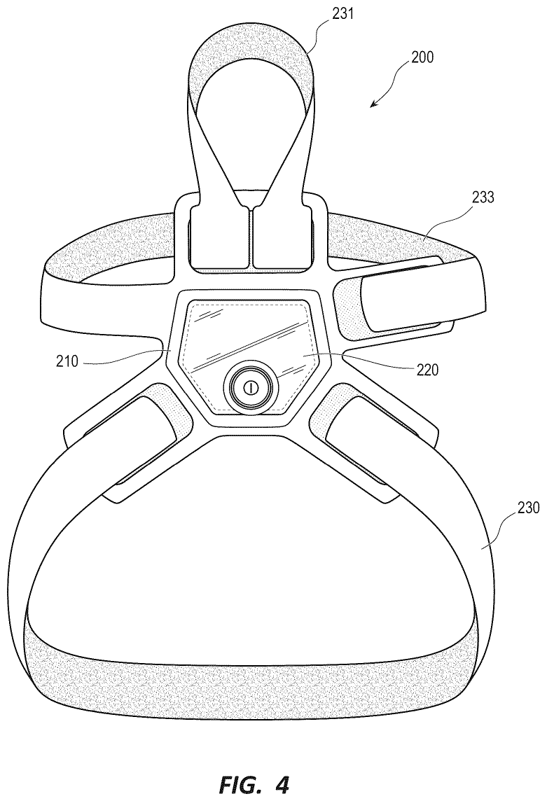

FIGS. 4-7B depict an embodiment of a hemostasis device 200 that resembles the hemostasis device 100 described above in certain respects. Accordingly, like features are designated with like reference numerals, with the leading digits incremented to “2.” For example, the embodiment depicted in FIGS. 4-7B includes a compression member 220 that may, in some respects, resemble the compression member 120 of FIG. 1. Relevant disclosure set forth above regarding similarly identified features thus may not be repeated hereafter. Moreover, specific features of the hemostasis device 100 and related components shown in FIGS. 1-3B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the hemostasis device 200 and related components depicted in FIGS. 4-7B. Any suitable combination of the features, and variations of the same, described with respect to the hemostasis device 100 and related components illustrated in FIGS. 1-3B can be employed with the hemostasis device 200 and related components of FIGS. 4-7B, and vice versa. This pattern of disclosure applies equally to further embodiments depicted in subsequent figures and described hereafter, wherein the leading digits may be further incremented.

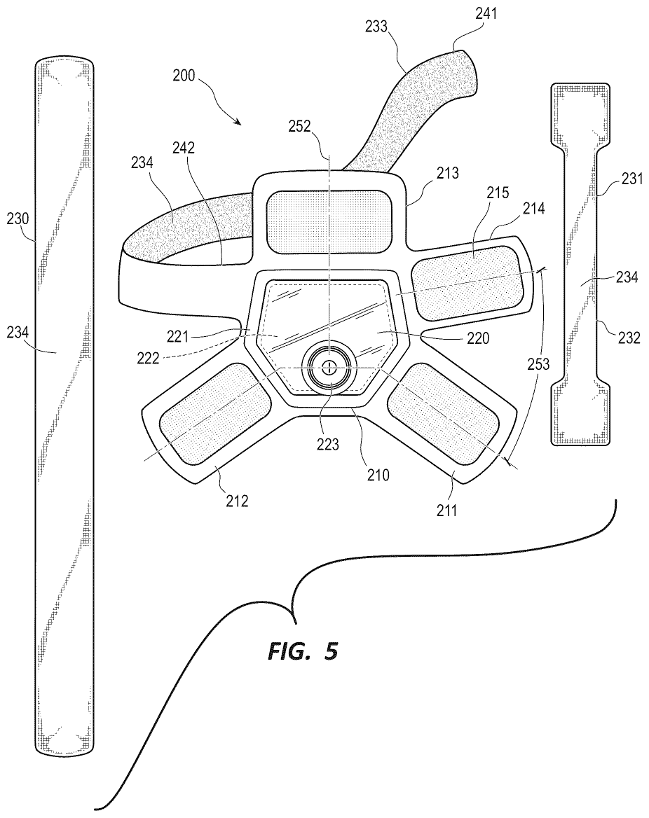

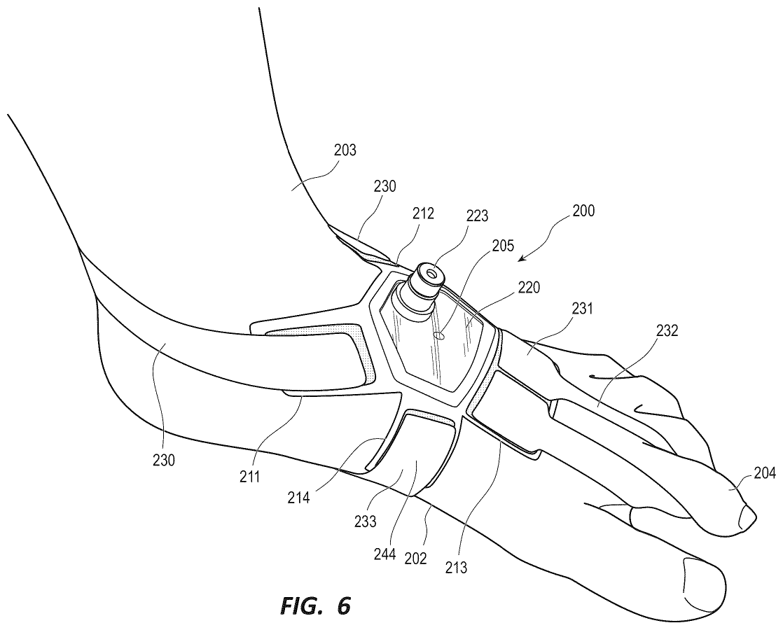

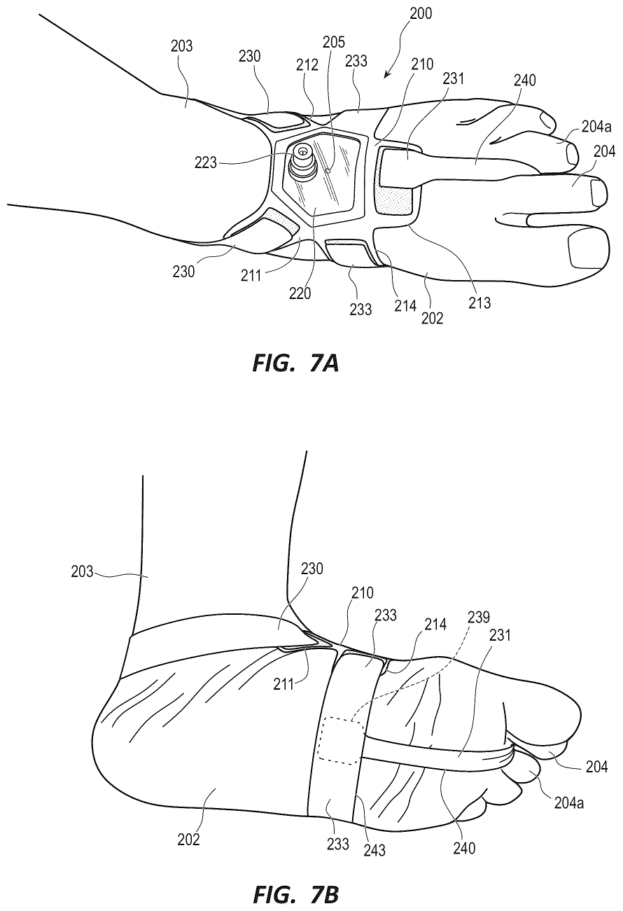

FIG. 4 provides a top view of a vascular access hemostasis device, system, or assembly 200. FIG. 5 provides a top, exploded view of the hemostasis device 200. FIG. 6 provides a perspective view of the hemostasis device 200 in a use configuration. FIGS. 7A and 7B provide a perspective view of the hemostasis device 200 in an alternative use configuration. As illustrated in the embodiment of FIG. 4, the hemostasis device 200 includes a body 210, a compression member 220, an ankle band or strap 230, a toe band or strap 231, and a foot band or strap 233 configured to wrap around a patient's foot. The hemostasis device 200 may include a first coupling tab 211, a second coupling tab 212, and a third coupling tab 213, which may be disposed a the same or different relative positions and angles as the coupling tabs 111, 112, 113 of the embodiment of FIGS. 1-3B.

As illustrated in FIG. 5, the body 210 includes a fourth coupling tab 214 extending radially outward and disposed between the first coupling tab 211 and the third coupling tab 213. An angle 253 between the first coupling tab 211 and the fourth coupling tab 214 can range from about 30 degrees to about 90 degrees. The fourth coupling tab 214 includes a releasable coupling member 215, such as a “hook” portion of a “hook-and-loop” material. The foot band 233 includes a releasable coupling member 234, such as a “loop” portion of the “hook-and-loop” material. A first end portion 241 is releasably couplable to the fourth coupling tab 214. In the illustrated embodiment, a second end portion 242 is fixedly coupled to the body 210 between the second coupling tab 212 and the third coupling tab 313. In certain embodiments, the foot band 233 and the body 210 may include a unibody construction. In other embodiments, the body 210 may include a fifth coupling tab, and the second end portion 242 may be releasably couplable to the fifth coupling tab.

As with the embodiment of FIGS. 1-3B, hemostasis device 200 of FIG. 5 may be configured such that any of the bands 230, 231, 233 is fixedly coupled to the body 210 at one or both ends and/or embodiments wherein any of the bands 230, 231, 233 are releasably coupleable to the body 210 at one or both ends. As also discussed in connection with the embodiment of FIGS. 1-3B, in embodiments wherein an end of any of the bands 230, 231, 233 is releasably coupleable to any of the coupling tabs 211, 212, 213, 214 (or any other portion of the body) the angle at which the band 230, 231, 233 couples to the body may be adjustable by a practitioner during use.

In use, as shown in FIG. 6, the compression member 220 of the hemostasis device 200 may be positioned over the vascular access puncture site 205 at a dorsal or medial aspect of a patient's foot 202. The ankle band 230 may be extended around the patient's ankle 203 and be releasably coupled to the first and second coupling tabs 211, 212 to restrict distal movement of the hemostasis device 200. The toe band 231 may extend toward and be wrapped around any one of the patient's toes 204 and be releasably coupled to the third coupling tab 213 to restrict proximal movement of the hemostasis device 100. Alternatively, as shown in FIGS. 7A and 7B, the toe band 231 may extend toward the patient's toes 204 and pass between adjacent toes 204, 204a (embodiments wherein the toe band 231 is configured to pass between any of the patients toes are within the scope of this disclosure as well). The second end portion 239 may be releasably coupled to an intermediate portion 243 of the foot band 233 at a bottom surface of the patient's foot 202. The foot band 233 is wrapped around the patient's foot 202 and the first end 241 is releasably coupled to the fourth coupling tab 214 to provide a downward force to the hemostasis device 200.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of applying a compressive force to a vascular access puncture site may include one or more of the following steps: positioning a compression member of a hemostasis device over a vascular access puncture site; wrapping a first securement band around an ankle or foot of a patient; releasably coupling a first end of the first securement band to a first coupling tab of the hemostasis device; releasably coupling a second end of the first securement band to a second coupling tab of the hemostasis device; extending a second securement band around a toe or between adjacent toes of the patient; releasably coupling a first end of the second securement band to a third coupling tab of the hemostasis device; releasably coupling a second end of the second securement band to the third coupling tab of the hemostasis device; and inflating the compression member to apply a compressive force to the vascular access puncture site. Other steps are also contemplated.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

“Fluid” is used in its broadest sense, to refer to any fluid, including both liquids and gases as well as solutions, compounds, suspensions, etc., which generally behave as fluids.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

The terms “a” and “an” can be described as one, but not limited to one. For example, although the disclosure may recite a housing having “a stopper,” the disclosure also contemplates that the housing can have two or more stoppers.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1-13. (canceled)

14. A method of applying a compressive force to a vascular access puncture site, comprising:
positioning a compression member of a hemostasis device over a vascular access puncture site;
wrapping a first securement band around an ankle or foot of a patient;
releasably coupling a first end of the first securement band to a first coupling tab of the hemostasis device;
releasably coupling a second end of the first securement band to a second coupling tab of the hemostasis device;
extending a second securement band around a toe or between adjacent toes of the patient;
releasably coupling a first end of the second securement band to a third coupling tab of the hemostasis device;
releasably coupling a second end of the second securement band to the third coupling tab of the hemostasis device; and
inflating the compression member to apply a compressive force to the vascular access puncture site.

15. The method of claim 14, further comprising:
wrapping a third securement band around the foot of the patient; and
releasably coupling a first end of the third securement band to a fourth tab of the hemostasis device.

16. A compression hemostasis device, comprising:
a body comprising:
a first coupling tab;
a second coupling tab;
a third coupling tab;
a fourth coupling tab circumferentially disposed between the first coupling tab and the third coupling tab; and
a compression member configured to compress a vascular puncture site;
a plurality of securement bands, comprising:
a first securement band releasably coupled to the first and second coupling tabs and configured to wrap around an ankle of a patient;
a second securement band releasably coupled to the third tab and configured to wrap around the patient's toe or extend between adjacent toes of the patient; and
a third securement band releasably coupled to the fourth coupling tab and fixedly coupled to the body circumferentially between the first coupling tab and the third coupling tab, wherein the third securement band is configured to wrap around a foot of the patient.

17. The hemostasis device of claim 16,
wherein an angle between the first coupling tab and the second coupling tab ranges from 60 degrees to 120 degrees;
wherein an angle between the first coupling tab and the third coupling tab ranges from 90 degrees to 150 degrees; and
wherein an angle between the first coupling tab and the fourth coupling tab ranges from 30 degrees to 90 degrees.

18. The hemostasis device of claim 17, wherein the second securement band is releasably coupled to the third securement band.

19. The hemostasis device of claim 17, wherein the compression member comprises:
a top plate;
an inflatable bladder coupled to a bottom surface of the top plate; and
an inflation port in fluid communication with the inflatable bladder.

20. The hemostasis device of claim 19, wherein the inflation port comprises a self-closing valve member coupled to the top plate.

21. The hemostasis device of claim 16, wherein each of the first, second, third, and fourth coupling tabs comprises a hook portion of a hook-and-loop material.

22. The hemostasis device of claim 16, wherein each of the first, second, and third securement bands comprises a loop portion of a hook-and-loop material.

23. The hemostasis device of claim 16, wherein the second securement band comprises a narrowed middle portion disposed between wide end portions.

24. The hemostasis device of claim 16, wherein the third securement band comprises a loop portion of a hook-and-loop material.

25. The hemostasis device of claim 16, wherein the second securement band comprises a hook portion of a hook-and-loop material.

26. The hemostasis device of claim 16, wherein the inflatable bladder is configured to apply a compressive force to the puncture site when inflated.

27. The hemostasis device of claim 16, wherein the inflatable bladder is configured to be inflated with air and/or a liquid.

28. The hemostasis device of claim 16, wherein the vascular access puncture site comprises any one of an anterior tibial artery, dorsalis pedis artery, posterior tibial artery, and peroneal artery.

29. The method of claim 14, wherein inflating the compression member comprises injecting air or liquid into an inflatable bladder of the compression member.

30. The method of claim 14, wherein the vascular access puncture site comprises any one of an anterior tibial artery, dorsalis pedis artery, posterior tibial artery, and peroneal artery.
